SEQUENCE LISTING (28 sequences)

SEQ ID NO: 1 - DNA, 73 bp, Artificial sequence
  Single strand DNA oligonucleotide
  misc_feature (21)..(53): n is a, c, g, or t
ttaagaaaatccttgcttaannnnnnnnnnnnnnnnnnnnnnnnnnnnnnnnnaaagattctctttttttatg

SEQ ID NO: 2 - DNA, 50 bp, Artificial sequence
  Single strand DNA oligonucleotide
ccgaagtcggtgatgctgtcgccgaagaagttaagaaaatccttgcttaa

SEQ ID NO: 3 - DNA, 50 bp, Artificial sequence
  Single strand DNA oligonucleotide
atttcatttataaagtttatgtacaaatatcataaaaaaagagaatcttt

SEQ ID NO: 4 - DNA, 1861 bp, Artificial sequence
  LEU2 nucleic acid sequence
aactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagagcgccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatatttcaaggatataccattctaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgttccacttccagatgaggcgctggaagcctccaagaaggctgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggtcaaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatgtaaaggaatacaggtaagcaaattgatactaatggctcaacgtgataaggaaaaagaattgcactttaacattaatattgacaaggaggagggcaccacacaaaaagttaggtgtaacagaaaatcatgaaactatgattcctaatttatatattggaggattttctctaaaaaaaaaaaaatacaacaaataaaaaacac

SEQ ID NO: 5 - DNA, 675 bp, Artificial sequence
  TRP1 nucleic acid sequence
atgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaatag

SEQ ID NO: 6 - DNA, 1716 bp, Artificial sequence
  ADE2 nucleic acid sequence
atggattctagaacagttggtatattaggagggggacaattgggacgtatgattgttgaggcagcaaacaggctcaacattaagacggtaatactagatgctgaaaattctcctgccaaacaaataagcaactccaatgaccacgttaatggctccttttccaatcctcttgatatcgaaaaactagctgaaaaatgtgatgtgctaacgattgagattgagcatgttgatgttcctacactaaagaatcttcaagtaaaacatcccaaattaaaaatttacccttctccagaaacaatcagattgatacaagacaaatatattcaaaaagagcatttaatcaaaaatggtatagcagttacccaaagtgttcctgtggaacaagccagtgagacgtccctattgaatgttggaagagatttgggttttccattcgtcttgaagtcgaggactttggcatacgatggaagaggtaacttcgttgtaaagaataaggaaatgattccggaagctttggaagtactgaaggatcgtcctttgtacgccgaaaaatgggcaccatttactaaagaattagcagtcatgattgtgagatctgttaacggtttagtgttttcttacccaattgtagagactatccacaaggacaatatttgtgacttatgttatgcgcctgctagagttccggactccgttcaacttaaggcgaagttgttggcagaaaatgcaatcaaatcttttcccggttgtggtatatttggtgtggaaatgttctatttagaaacaggggaattgcttattaacgaaattgccccaaggcctcacaactctggacattataccattgatgcttgcgtcacttctcaatttgaagctcatttgagatcaatattggatttgccaatgccaaagaatttcacatctttctccaccattacaacgaacgccattatgctaaatgttcttggagacaaacatacaaaagataaagagctagaaacttgcgaaagagcattggcgactccaggttcctcagtgtacttatatggaaaagagtctagacctaacagaaaagtaggtcacataaatattattgcctccagtatggcggaatgtgaacaaaggctgaactacattacaggtagaactgatattccaatcaaaatctctgtcgctcaaaagttggacttggaagcaatggtcaaaccattggttggaatcatcatgggatcagactctgacttgccggtaatgtctgccgcatgtgcggttttaaaagattttggcgttccatttgaagtgacaatagtctctgctcatagaactccacataggatgtcagcatatgctatttccgcaagcaagcgtggaattaaaacaattatcgctggagctggtggggctgctcacttgccaggtatggtggctgcaatgacaccacttcctgtcatcggtgtgcccgtaaaaggttcttgtctagatggagtagattctttacattcaattgtgcaaatgcctagaggtgttccagtagctaccgtcgctattaataatagtacgaacgctgcgctgttggctgtcagactgcttggcgcttatgattcaagttatacaacgaaaatggaacagtttttattaaagcaagaagaagaagttcttgtcaaagcacaaaagttagaaactgtcggttacgaagcttatctagaaaacaagtaa

SEQ ID NO: 7 - DNA, 4179 bp, Artificial sequence
  LYS2 nucleic acid sequence
atgactaacgaaaaggtctggatagagaagttggataatccaactctttcagtgttaccacatgactttttacgcccacaacaagaaccttatacgaaacaagctacatattcgttacagctacctcagctcgatgtgcctcatgatagtttttctaacaaatacgctgtcgctttgagtgtatgggctgcattgatatatagagtaaccggtgacgatgatattgttctttatattgcgaataacaaaatcttaagattcaatattcaaccaacgtggtcatttaatgagctgtattctacaattaacaatgagttgaacaagctcaattctattgaggccaatttttcctttgacgagctagctgaaaaaattcaaagttgccaagatctggaaaggacccctcagttgttccgtttggcctttttggaaaaccaagatttcaaattagacgagttcaagcatcatttagtggactttgctttgaatttggataccagtaataatgcgcatgttttgaacttaatttataacagcttactgtattcgaatgaaagagtaaccattgttgcggaccaatttactcaatatttgactgctgcgctaagcgatccatccaattgcataactaaaatctctctgatcaccgcatcatccaaggatagtttacctgatccaactaagaacttgggctggtgcgatttcgtggggtgtattcacgacattttccaggacaatgctgaagccttcccagagagaacctgtgttgtggagactccaacactaaattccgacaagtcccgttctttcacttatcgcgacatcaaccgcacttctaacatagttgcccattatttgattaaaacaggtatcaaaagaggtgatgtagtgatgatctattcttctaggggtgtggatttgatggtatgtgtgatgggtgtcttgaaagccggcgcaaccttttcagttatcgaccctgcatatcccccagccagacaaaccatttacttaggtgttgctaaaccacgtgggttgattgttattagagctgctggacaattggatcaactagtagaagattacatcaatgatgaattggagattgtttcaagaatcaattccatcgctattcaagaaaatggtaccattgaaggtggcaaattggacaatggcgaggatgttttggctccatatgatcactacaaagacaccagaacaggtgttgtagttggaccagattccaacccaaccctatctttcacatctggttccgaaggtattcctaagggtgttcttggtagacatttttccttggcttattatttcaattggatgtccaaaaggttcaacttaacagaaaatgataaattcacaatgctgagcggtattgcacatgatccaattcaaagagatatgtttacaccattatttttaggtgcccaattgtatgtccctactcaagatgatattggtacaccgggccgtttagcggaatggatgagtaagtatggttgcacagttacccatttaacacctgccatgggtcaattacttactgcccaagctactacaccattccctaagttacatcatgcgttctttgtgggtgacattttaacaaaacgtgattgtctgaggttacaaaccttggcagaaaattgccgtattgttaatatgtacggtaccactgaaacacagcgtgcagtttcttatttcgaagttaaatcaaaaaatgacgatccaaactttttgaaaaaattgaaagatgtcatgcctgctggtaaaggtatgttgaacgttcagctactagttgttaacaggaacgatcgtactcaaatatgtggtattggcgaaataggtgagatttatgttcgtgcaggtggtttggccgaaggttatagaggattaccagaattgaataaagaaaaatttgtgaacaactggtttgttgaaaaagatcactggaattatttggataaggataatggtgaaccttggagacaattctggttaggtccaagagatagattgtacagaacgggtgatttaggtcgttatctaccaaacggtgactgtgaatgttgcggtagggctgatgatcaagttaaaattcgtgggttcagaatcgaattaggagaaatagatacgcacatttcccaacatccattggtaagagaaaacattactttagttcgcaaaaatgccgacaatgagccaacattgatcacatttatggtcccaagatttgacaagccagatgacttgtctaagttccaaagtgatgttccaaaggaggttgaaactgaccctatagttaagggcttaatcggttaccatcttttatccaaggacatcaggactttcttaaagaaaagattggctagctatgctatgccttccttgattgtggttatggataaactaccattgaatccaaatggtaaagttgataagcctaaacttcaattcccaactcccaagcaattaaatttggtagctgaaaatacagtttctgaaactgacgactctcagtttaccaatgttgagcgcgaggttagagacttatggttaagtatattacctaccaagccagcatctgtatcaccagatgattcgtttttcgatttaggtggtcattctatcttggctaccaaaatgatttttaccttaaagaaaaagctgcaagttgatttaccattgggcacaattttcaagtatccaacgataaaggcctttgccgcggaaattgacagaattaaatcatcgggtggatcatctcaaggtgaggtcgtcgaaaatgtcactgcaaattatgcggaagacgccaagaaattggttgagacgctaccaagttcgtacccctctcgagaatattttgttgaacctaatagtgccgaaggaaaaacaacaattaatgtgtttgttaccggtgtcacaggatttctgggctcctacatccttgcagatttgttaggacgttctccaaagaactacagtttcaaagtgtttgcccacgtcagggccaaggatgaagaagctgcatttgcaagattacaaaaggcaggtatcacctatggtacttggaacgaaaaatttgcctcaaatattaaagttgtattaggcgatttatctaaaagccaatttggtctttcagatgagaagtggatggatttggcaaacacagttgatataattatccataatggtgcgttagttcactgggtttatccatatgccaaattgagggatccaaatgttatttcaactatcaatgttatgagcttagccgccgtcggcaagccaaagttctttgactttgtttcctccacttctactcttgacactgaatactactttaatttgtcagataaacttgttagcgaagggaagccaggcattttagaatcagacgatttaatgaactctgcaagcgggctcactggtggatatggtcagtccaaatgggctgctgagtacatcattagacgtgcaggtgaaaggggcctacgtgggtgtattgtcagaccaggttacgtaacaggtgcctctgccaatggttcttcaaacacagatgatttcttattgagatttttgaaaggttcagtccaattaggtaagattccagatatcgaaaattccgtgaatatggttccagtagatcatgttgctcgtgttgttgttgctacgtctttgaatcctcccaaagaaaatgaattggccgttgctcaagtaacgggtcacccaagaatattattcaaagactacttgtatactttacacgattatggttacgatgtcgaaatcgaaagctattctaaatggaagaaatcattggaggcgtctgttattgacaggaatgaagaaaatgcgttgtatcctttgctacacatggtcttagacaacttacctgaaagtaccaaagctccggaactagacgataggaacgccgtggcatctttaaagaaagacaccgcatggacaggtgttgattggtctaatggaataggtgttactccagaagaggttggtatatatattgcatttttaaacaaggttggatttttacctccaccaactcataatgacaaacttccactgccaagtatagaactaactcaagcgcaaataagtctagttgcttcaggtgctggtgctcgtggaagctccgcagcagcttaa

SEQ ID NO: 8 - DNA, 33 bp, Artificial sequence
  33 BP FRAGMENT INSERTED IN LEU2 REPORTE
ataactttgcaacagtgaaggaaagcctttgga

SEQ ID NO: 9 - DNA, 6692 bp, Artificial sequence
  ADAR nucleic acid sequence
gcccctcctcttggccaaactttccggaggggaaggctttccgaggaaacgaaagcgaaattgaaccggagccatcttgggcccggcgcgcagacccgcggagtttcccgtgccgacgccccggggccacttccagtgcggagtagcggaggcgtgggggcctcgaggggctggcgcggcccagcggtcgggccagggtcgtgccgccggcgggtcgggccgggcaatgcctcgcgggcgcaatgaatccgcggcaggggtattccctcagcggatactacacccatccatttcaaggctatgagcacagacagctcaggtaccagcagcctgggccaggatcttcccccagtagtttcctgcttaagcaaatagaatttctcaaggggcagctcccagaagcaccggtgattggaaagcagacaccgtcactgccaccttccctcccaggactccggccaaggtttccagtactacttgcctccagtaccagaggcaggcaagtggacatcaggggtgtccccaggggcgtgcatctcggaagtcaggggctccagagagggttccagcatccttcaccacgtggcaggagtctgccacagagaggtgttgattgcctttcctcacatttccaggaactgagtatctaccaagatcaggaacaaaggatcttaaagttcctggaagagcttggggaagggaaggccaccacagcacatgatctgtctgggaaacttgggactccgaagaaagaaatcaatcgagttttatactccctggcaaagaagggcaagctacagaaagaggcaggaacaccccctttgtggaaaatcgcggtctccactcaggcttggaaccagcacagcggagtggtaagaccagacggtcatagccaaggagccccaaactcagacccgagtttggaaccggaagacagaaactccacatctgtctcagaagatcttcttgagccttttattgcagtctcagctcaggcttggaaccagcacagcggagtggtaagaccagacagtcatagccaaggatccccaaactcagacccaggtttggaacctgaagacagcaactccacatctgccttggaagatcctcttgagtttttagacatggccgagatcaaggagaaaatctgcgactatctcttcaatgtgtctgactcctctgccctgaatttggctaaaaatattggccttaccaaggcccgagatataaatgctgtgctaattgacatggaaaggcagggggatgtctatagacaagggacaacccctcccatatggcatttgacagacaagaagcgagagaggatgcaaatcaagagaaatacgaacagtgttcctgaaaccgctccagctgcaatccctgagaccaaaagaaacgcagagttcctcacctgtaatatacccacatcaaatgcctcaaataacatggtaaccacagaaaaagtggagaatgggcaggaacctgtcataaagttagaaaacaggcaagaggccagaccagaaccagcaagactgaaaccacctgttcattacaatggcccctcaaaagcagggtatgttgactttgaaaatggccagtgggccacagatgacatcccagatgacttgaatagtatccgcgcagcaccaggtgagtttcgagccatcatggagatgccctccttctacagtcatggcttgccacggtgttcaccctacaagaaactgacagagtgccagctgaagaaccccatcagcgggctgttagaatatgcccagttcgctagtcaaacctgtgagttcaacatgatagagcagagtggaccaccccatgaacctcgatttaaattccaggttgtcatcaatggccgagagtttcccccagctgaagctggaagcaagaaagtggccaagcaggatgcagctatgaaagccatgacaattctgctagaggaagccaaagccaaggacagtggaaaatcagaagaatcatcccactattccacagagaaagaatcagagaagactgcagagtcccagacccccaccccttcagccacatccttcttttctgggaagagccccgtcaccacactgcttgagtgtatgcacaaattggggaactcctgcgaattccgtctcctgtccaaagaaggccctgcccatgaacccaagttccaatactgtgttgcagtgggagcccaaactttccccagtgtgagtgctcccagcaagaaagtggcaaagcagatggccgcagaggaagccatgaaggccctgcatggggaggcgaccaactccatggcttctgataaccagcctgaaggtatgatctcagagtcacttgataacttggaatccatgatgcccaacaaggtcaggaagattggcgagctcgtgagatacctgaacaccaaccctgtgggtggccttttggagtacgcccgctcccatggctttgctgctgaattcaagttggtcgaccagtccggacctcctcacgagcccaagttcgtttaccaagcaaaagttgggggtcgctggttcccagccgtctgcgcacacagcaagaagcaaggcaagcaggaagcagcagatgcggctctccgtgtcttgattggggagaacgagaaggcagaacgcatgggtttcacagaggtaaccccagtgacaggggccagtctcagaagaactatgctcctcctctcaaggtccccagaagcacagccaaagacactccctctcactggcagcaccttccatgaccagatagccatgctgagccaccggtgcttcaacactctgactaacagcttccagccctccttgctcggccgcaagattctggccgccatcattatgaaaaaagactctgaggacatgggtgtcgtcgtcagcttgggaacagggaatcgctgtgtgaaaggagattctctcagcctaaaaggagaaactgtcaatgactgccatgcagaaataatctcccggagaggcttcatcaggtttctctacagtgagttaatgaaatacaactcccagactgcgaaggatagtatatttgaacctgctaagggaggagaaaagctccaaataaaaaagactgtgtcattccatctgtatatcagcactgctccgtgtggagatggcgccctctttgacaagtcctgcagcgaccgtgctatggaaagcacagaatcccgccactaccctgtcttcgagaatcccaaacaaggaaagctccgcaccaaggtggagaacggagaaggcacaatccctgtggaatccagtgacattgtgcctacgtgggatggcattcggctcggggagagactccgtaccatgtcctgtagtgacaaaatcctacgctggaacgtgctgggcctgcaaggggcactgttgacccacttcctgcagcccatttatctcaaatctgtcacattgggttaccttttcagccaagggcatctgacccgtgctatttgctgtcgtgtgacaagagatgggagtgcatttgaggatggactacgacatccctttattgtcaaccaccccaaggttggcagagtcagcatatatgattccaaaaggcaatccgggaagactaaggagacaagcgtcaactggtgtctggctgatggctatgacctggagatcctggacggtaccagaggcactgtggatgggccacggaatgaattgtcccgggtctccaaaaagaacatttttcttctatttaagaagctctgctccttccgttaccgcagggatctactgagactctcctatggtgaggccaagaaagctgcccgtgactacgagacggccaagaactacttcaaaaaaggcctgaaggatatgggctatgggaactggattagcaaaccccaggaggaaaagaacttttatctctgcccagtatagtatgctccagtgacagatggattagggtgtgtcatactagggtgtgagagaggtaggtcgtagcattcctcatcacatggtcaggggatttttttttctcctttttttttctttttaagccataattggtgatactgaaaactttgggttcccatttatcctgctttctttgggattgctaggcaaggtctggccaggccccccttttttcccccaagtgaagaggcagaaacctaagaagttatcttttctttctacccaaagcatacatagtcactgagcacctgcggtccatttcctcttaaaagttttgttttgatttgtttccatttcctttccctttgtgtttgctacactgacctcttgcggtcttgattaggtttcagtcaactctggatcatgtcagggactgataatttcatttgtggattacgcagacccctctacttcccctctttcccttctgagattctttccttgtgatctgaatgtctccttttccccctcagagggcaaagaggtgaacataaaggatttggtgaaacatttgtaagggtaggagttgaaaactgcagttcccagtgccacggaagtgtgattggagcctgcagataatgcccagccatcctcccatcctgcactttagccagctgcagggcgggcaaggcaaggaaagctgcttccctggaagtgtatcactttctccggcagctgggaagtctagaaccagccagactgggttaagggagctgctcaagcaatagcagaggtttcacccggcaggatgacacagaccacttcccagggagcacgggcatgccttggaatattgccaagcttccagctgcctcttctcctaaagcattcctaggaatattttccccgccaatgctgggcgtacaccctagccaacgggacaaatcctagagggtataaaatcatctctgctcagataatcatgacttagcaagaataagggcaaaaaatcctgttggcttaacgtcactgttccacccggtgtaatatctctcatgacagtgacaccaagggaagttgactaagtcacatgtaaattaggagtgttttaaagaatgccatagatgttgattcttaactgctacagataacctgtaattgagcagatttaaaattcaggcatacttttccatttatccaagtgctttcatttttccagatggcttcagaagtaggctcgtgggcagggcgcagacctgatctttatagggttgacatagaaagcagtagttgtgggtgaaagggcaggttgtcttcaaactctgtgaggtagaatcctttgtctatacctccatgaacattgactcgtgtgttcagagcctttggcctctctgtggagtctggctctctggctcctgtgcattctttgaatagtcactcgtaaaaactgtcagtgcttgaaactgtttcctttactcatgttgaagggactttgttggcttttagagtgttggtcatgactccaagagcagagcagggaagagcccaagcatagacttggtgccgtggtgatggctgcagtccagttttgtgatgctgcttttacgtgtccctcgataacagtcagctagacacactcaggaggactactgaggctctgcgaccttcaggagctgagcctgcctctctcctttagatgacagaccttcatctgggaacgtgctgagccagcaccctcagatgatttccctccaaactgctgactaggtcatcctctgtctggtagagacattcacatctttgcttttattctatgctctctgtacttttgaccaaaaattgaccaaagtaagaaaatgcaagttctaaaaatagactaaggatgcctttgcagaacaccaaagcatcccaaggaactggtagggaagtggcgcctgtctcctggagtggaagaggcctgctccctggctctgggtctgctgggggcacagtaaatcagtcttggcacccacatccagggcagagaggtctgtggttctcagcatcagaaggcagcgcagcccctctcctcttcaggctacagggttgtcacctgctgagtcctcaggttgtttggcctctctggtccatcttgggcattaggttctccagcagagctctggccagctgcctcttctttaactgggaacacaggctctcacaagatcagaacccccactcacccccaagatcttatctagcaagcctgtagtattcagtttctgttgtaggaagagagcgaggcatccctgaattccacgcatctgctggaaacgagccgtgtcagatcgcacatccctgcgcccccatgcccctctgagtcacacaggacagaggaggcagagcttctgcccactgttatcttcactttctttgtccagtcttttgtttttaataagcagtgaccctccctactcttctttttaatgatttttgtagttgatttgtctgaactgtggctactgtgcattccttgaataatcacttgtaaaaattgtcagtgcttgaagctgtttcctttactcacattgaagggacttcgttggttttttggagtcttggttgtgactccaagagcagagtgaggaagacccccaagcatagactcgggtactgtgatgatggctgcagtccagttttatgattctgcttttatgtgtcccttgataacagtgacttaacaatatacattcctcataaataaaaaaaaaacaagaatctgaattcttagaaaaaaaaaaaaaaaaaaaaaaa

SEQ ID NO: 10 - DNA, 33 bp, Artificial sequence
  mutated Leu2 partial sequence (leu2W82X)
gtgggtggtcctaaatagggtaccggtagtgtt

SEQ ID NO: 11 - DNA, 33 bp, Artificial sequence
  mutated Leu2 partial sequence (leu2W82X)
cacccaccaggatttatcccatggccatcacaa

SEQ ID NO: 12 - protein, 5 aa, Artificial sequence
  mutated Leu2 partial sequence (leu2W82X)
VGGPK

SEQ ID NO: 13 - DNA, 33 bp, Artificial sequence
  wild-type Leu2 partial sequence
aacactaccggtaccccatttaggaccacccac

SEQ ID NO: 14 - DNA, 33 bp, Artificial sequence
  wild-type Leu2 partial sequence
ttgtgatggccatggggtaaatcctggtgggtg

SEQ ID NO: 15 - protein, 11 aa, Artificial sequence
  wild-type Leu2 partial sequence
VSGTGWKPGGV

SEQ ID NO: 16 - DNA, 46 bp, Artificial sequence
  Tail nucleic acid sequence sequence
aatccttgcttaaaacactaccggtaccccatttaggaccacccac

SEQ ID NO: 17 - DNA, 46 bp, Artificial sequence
  Tail nucleic acid sequence sequence
aatccttgcttaaaacactaccggtatcccatttaggaccacccac

SEQ ID NO: 18 - DNA, 46 bp, Artificial sequence
  Tail nucleic acid sequence sequence
aatccttgcttaaaacactaccggtaccccatttaggccgacccac

SEQ ID NO: 19 - DNA, 46 bp, Artificial sequence
  Tail nucleic acid sequence sequence
aatccttgcttaaaacactactggtaccccatttagggccacccac

SEQ ID NO: 20 - DNA, 46 bp, Artificial sequence
  Tail nucleic acid sequence sequence
aatccttgcttaaaacactaccggtttcccatttaggaccacccac

SEQ ID NO: 21 - DNA, 34 bp, Artificial sequence
  mutated Leu2-CF partial sequence (leu2W82X)
ataactttgcaacagtgaaggaaagcctttggat

SEQ ID NO: 22 - DNA, 34 bp, Artificial sequence
  mutated Leu2-CF partial sequence (leu2W82X)
tattgaaacgttgtcacttcctttcggaaaccta

SEQ ID NO: 23 - protein, 5 aa, Artificial sequence
  mutated Leu2-CF partial sequence (leu2W82X)
ITLQQ

SEQ ID NO: 24 - DNA, 33 bp, Artificial sequence
  wild-type Leu2-CF partial sequence
tccaaaggctttcctccactgttgcaaagttat

SEQ ID NO: 25 - DNA, 33 bp, Artificial sequence
  wild-type Leu2-CF partial sequence
aggtttccgaaaggaggtgacaacgtttcaata

SEQ ID NO: 26 - protein, 11 aa, Artificial sequence
  wild-type Leu2-CF partial sequence
GFAKRWQQLTI

SEQ ID NO: 27 - protein, 5 aa, Artificial sequence
  mutated Leu2 partial sequence (leu2W82X)
GTGSV

SEQ ID NO: 28 - protein, 6 aa, Artificial sequence
  mutated Leu2-CF partial sequence (leu2W82X)
RRKAFG

(19) United States
(12) Patent Application Publication
(10) Pub. No.: US 2023/0242906 A1
(43) Pub. Date: Aug. 3, 2023

(54) COMPOSITION AND METHODS FOR IDENTIFYING ANTISENSE GUIDE RNA FOR RNA EDITING

(71) Applicant: Bar-Ilan University, Ramat-Gan (IL)

(72) Inventors: Erez LEVANON, Petach-Tikva (IL); Shay BEN-AROYA, Kiryat-Ono (IL)

(73) Assignee: Bar-Ilan University, Ramat-Gan (IL)

(21) Appl. No.: 18/013,628

(22) PCT Filed: Jun. 29, 2021
(86) PCT No.: PCT/IL2021/050800

§ 371 Date: Dec. 29, 2022

Related U.S. Application Data
(60) Provisional application No. 63/045,216, filed on Jun. 29, 2020.

(51) Int. Cl. C12N 15/10; C12Q 1/6897; C12N 15/113; C12N 15/81

(52) U.S. Cl. CPC C12N 15/1086; C12Q 1/6897; C12N 15/113; C12N 15/81; C12N 2310/11

(57) ABSTRACT

Compositions and methods for identifying antisense guide RNA for RNA editing are provided. Accordingly, there is provided a polynucleotide comprising: (i) a nucleic acid sequence encoding a reporter polypeptide comprising a heterologous nucleic acid sequence introducing an in-frame premature stop codon comprising an adenosine preventing translation of a functional reporter polypeptide; and (ii) an additional nucleic acid sequence heterologous to said reporter polypeptide having at least 60% complementarity to said nucleic acid sequence comprising said in-frame premature stop codon.

### RELATED APPLICATION/S

This application claims the benefit of priority of US Provisional Patent Application No. 63/045,216 filed on Jun. 29, 2020, the contents of which are incorporated herein by reference in their entirety.

### SEQUENCE LISTING STATEMENT

The ASCII file, entitled 88549Sequence Listing.txt, created on Jun. 29, 2021, comprising 28,672 bytes, submitted concurrently with the filing of this application is incorporated herein by reference.

### FIELD AND BACKGROUND OF THE INVENTION

The present invention, in some embodiments thereof, relates to compositions and methods for identifying antisense guide RNA for RNA editing.

RNA editing is a natural process through which eukaryotic cells alter the sequence of RNA molecules, often in a site-specific and precise way, thereby increasing the repertoire of genome encoded RNAs by several orders of magnitude. RNA editing enzymes have been described for eukaryotic species throughout the animal and plant kingdoms, and these processes play an important role in managing cellular homeostasis in metazoans from the simplest life forms to humans.

Unlike DNA editing, RNA editing manipulates genetic information in a reversible and tunable manner making it a promising target for therapeutics enabling manipulations that are either lethal or quickly compensated when done at the genome level. Furthermore, RNA editing could be safer because potential adverse effects and off-target edits should be reversible and dose-dependent.

The most abundant and studied form of RNA editing system in Metazoans is the adenosine deaminase enzyme, ADAR (adenosine deaminases acting on RNA). ADAR is a multi-domain protein, comprising a recognition domain and a catalytic domain. The recognition domain recognizes a specific dsRNA sequence and/or conformation, whereas the catalytic domain converts an adenosine into inosine in the target RNA, by deamination of the nucleobase. Inosine is read as guanine by the translational machinery of the cell, instead of the original adenosine that was encoded in the genome. Hence, Adenosine-to-inosine editing in RNA diversifies the transcriptome by recoding of amino acid codons, Start codons and Stop codons, and by alteration of splicing, among other mechanisms [Nishikura et al. Nat. Rev. Mol. Cell Biol. 17, 83-96 (2016)].

Steering ADAR to specific sites at selected transcripts, a strategy called site-directed RNA editing, holds great promise for the treatment of disease and as a tool to study protein and RNA function. An example comes with A's in stop codons (UGA,UAA, UAG) which, when edited, allow for read-through during translation;

thus, diseases caused by mutations that introduce termination codon (PTCs) can be corrected by RNA editing. Obviously, most A's do not occur within structures recognized by ADAR; therefore, several strategies have been developed to promote the editing of such targets. One such strategy, is to create substrates around a target A that are recognized by ADAR or an engineered ADAR. Essentially, these structures are generated by delivering antisense guide RNA oligos that create editable structures in trans. The proper design of these guides is critical. Currently, the most effective guide RNAs are composed of two essential elements: an antisense portion that is imperfectly complimentary to the mRNA in the vicinity of the targeted adenosine and a recruitment element to nucleate ADAR binding. Still, there are no generic rules for the construction of either element.

Additional Background Art Includes
International Patent Application Publication No. WO 2016097212;
Montiel-Gonzalez et al. Methods (2019) 156: 16-24;
Merlkle et al. Nature Biotechnology (2019) 37: 133-138;
Fukuda et al. Scientific Reports (2017) 7:41478;
Wettengel et al. Nucleic Acids Research (2017) 45(5): 2797-2808;
Wang et al. Biochemistry. (2018) 57(10): 1640-1651; and
Garncarz et al. RNA Biology (2013) 10:2, 192-204.

### SUMMARY OF THE INVENTION

According to an aspect of some embodiments of the present invention there is provided a polynucleotide comprising:
(i) a nucleic acid sequence encoding a reporter polypeptide comprising a heterologous nucleic acid sequence introducing an in-frame premature stop codon comprising an adenosine preventing translation of a functional reporter polypeptide; and
(ii) an additional nucleic acid sequence heterologous to the reporter polypeptide having at least 60% complementarity to the nucleic acid sequence comprising the in-frame premature stop codon;
   wherein the (i) and the (ii) are transcribed as a single transcript; and
   wherein conversion of the adenosine to inosine by RNA editing enables translation of a functional reporter polypeptide.

According to some embodiments of the invention, the reporter polypeptide is an auxotrophic polypeptide.

According to some embodiments of the invention, the auxotrophic polypeptide is selected from the group consisting of LEU2, TRP1, ADE2 and LYS2.

According to some embodiments of the invention, the reporter polypeptide confers resistance to an antibiotic.

According to some embodiments of the invention, the polypeptide conferring resistance to an antibiotic is selected from the group consisting of KanMX, NatMX and HygB.

According to some embodiments of the invention, the reporter polypeptide is LEU2.

According to some embodiments of the invention, the heterologous nucleic acid sequence introducing the in-frame premature stop codon is located between positions 244 and 246 corresponding to the LEU2 nucleic acid sequence as set forth in SEQ ID NO: 4.

According to some embodiments of the invention, the heterologous nucleic acid sequence introducing the in-frame premature stop codon is a specific nucleic acid sequence of a gene associated with a disease.

According to some embodiments of the invention, the d gene is selected from the group consisting of CFTR, LDLR, Factor IX, hexosaminidase and ATM.

According to some embodiments of the invention, the heterologous nucleic acid sequence introducing the in-frame premature stop codon is 15-120 nucleic acids long.

According to some embodiments of the invention, the at least 60% complementarity is at least 70% complementarity.

According to some embodiments of the invention, the at least 60% complementarity is at least 80% complementarity.

According to some embodiments of the invention, the (ii) comprises a mismatch with the adenosine.

According to some embodiments of the invention, the (ii) is 15-120 nucleic acids long.

According to some embodiments of the invention, the (i) is upstream of the (ii).

According to some embodiments of the invention, the polynucleotide being devoid of a nucleic acid linker between the (i) and the (ii).

According to some embodiments of the invention, the polynucleotide comprising (iii) an additional nucleic acid sequence encoding ADAR.

According to an aspect of some embodiments of the present invention there is provided a nucleic acid system comprising the polynucleotide and a polynucleotide comprising a nucleic acid sequence encoding ADAR.

According to some embodiments of the invention, the polynucleotide is comprised in a nucleic acid construct comprising a cis-acting regulatory element for directing expression of the polynucleotide,

According to an aspect of some embodiments of the present invention there is provided a cell expressing the polynucleotide or the system.

According to some embodiments of the invention, ADAR is capable of editing RNA in the cell.

According to some embodiments of the invention, the cell expresses an endogenous ADAR.

According to some embodiments of the invention, the cell does not express an endogenous ADAR.

According to some embodiments of the invention, the cell expresses an exogenous ADAR.

According to some embodiments of the invention, the cell is a eukaryotic cell.

According to some embodiments of the invention, the cell is a yeast cell.

According to some embodiments of the invention, the yeast is Saccharomyces cerevisiae.

According to an aspect of some embodiments of the present invention there is provided a method of identifying an antisense suitable for site-directed RNA editing, the method comprising determining in the cell translation of the functional reporter polypeptide, wherein when the cell is not expressing an ADAR capable of editing RNA in the cell the method comprises expressing in the cell a polynucleotide comprising a nucleic acid sequence encoding ADAR capable of editing RNA in the cell prior to the determining,

wherein the translation above a predetermined threshold indicates the (ii) is a suitable antisense for site-directed RNA editing of the in-frame premature stop codon.

According to some embodiments of the invention, the method being effected in-vitro or ex-vivo.

According to some embodiments of the invention, the method being effected in-vivo.

According to some embodiments of the invention, when the reporter polypeptide is an auxotrophic polypeptide or confers resistance to an antibiotic, the determining is effected by determining growth and/or survival under selective conditions.

According to some embodiments of the invention, the ADAR is human ADAR.

According to some embodiments of the invention, the ADAR is ADAR1.

According to some embodiments of the invention, the ADAR is ADAR2.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the Drawings:

FIG. 1 is a schematic representation of the yeast strain used in the selection system for directing ADAR activity towards the LEU2 gene. A leucine auxotroph yeast strain harbors a plasmid marked with the URA3 auxotrophic marker that can conditionally express human ADAR under a galactose inducible promoter (GAL1p-hADAR1). The endogenous LEU2 is deleted (leu2Δ) and replaced by a plasmid based LEU2, marked with the HIS3 auxotrophic marker. This exogenous LEU2 gene is dysfunctional as a result of a nonsense mutation (leu2W82X, denoted by a red bar in the plasmid). The 3′ end of the leu2W82X gene is followed by a replicable “tail” that can fold back at the RNA level to create dsRNA (denoted by a blue bar on the plasmid). An efficient RNA editing is expected to generate an amount of wild-type Leu2 protein sufficient enough to allow growth in a liquid medium without leucine.

FIGS. 2A-E demonstrate the yeast-based screening platform for identifying effective guide-RNAs for site-directed ADAR RNA editing. FIGS. 2A-B show schematic representations of the yeast-based screening platform. In FIG. 2A, the PCR products composed of different tails (denoted by colored rectangles) and the BamHI digested HIS3 plasmid described in FIG. 1 are co-transformed into the yeast cells carrying the URA3 marked GAL1-hADAR1 plasmid. Homologous recombination in yeast, and plating on a synthetic dropout (SD) medium lacking uracil and histidine (SD-URA-HIS), enables the selection of a library composed of 10⁸⁻10⁹ colonies, each containing a plasmid that is encircled by a different “tail” at the 3′ end of the engineered leu2W82X gene. In FIG. 2B, the random library described in FIG. 2A (represented by the colored yeast cells) is pooled. By applying selection (leucine starvation), cells are enriched for those carrying “tails” that promote efficient editing. Following, “tails” from the plasmids prepared from the pooled strains are PCR amplified in one pooled reaction, using a universal primer set that anneals to adjacent vector sequences, flanking the insertion site (black arrows); and their sequences are identified by DNA deep sequencing. FIGS. 2C-E show the results of a representative experiment for the selection of improved tail variants targeting the leu2W82X mutation. FIG. 2C shows images of the tubes containing the library of tails (right), and cells carrying tails that form perfect dsRNA structures with the leu2W82X target mutation (with an exception of a single mismatch between A and C at the STOP codon-containing sequence, left), following three iterative rounds of enrichment in a SC-GAL-URA-HIS-LEU medium. This medium is supplemented with 2% galactose (GAL) (to enable GAL1p-hADAR1 expression); and lacking uracil (hADAR1 plasmid selection), histidine (encircled HIS3 plasmid selection) and leucine (selection for hADAR1 mediated Leu2 protein synthesis). FIG. 2D shows growth curves of selected colonies formed by the single cells obtained from the samples described in FIG. 2C. The growth rate was compared to the intermediate growth-rate baseline of tails that form perfect dsRNA structures (denoted by a blue arrow). FIG. 2E shows sequence analysis of the “tails” supporting the growth of the colonies in FIG. 2D. Changes from the reference perfect dsRNA tail (highlighted by a green rectangle) are marked in red.

FIGS. 3A-B demonstrate the yeast-based screening platform for identifying effective guide-RNA for known CFTR nonsense mutants. FIG. 3A is a schematic representations of the screening platform, based on the system shown in FIG. 1 with the exception that a 33 bp fragment that contains the CFTR W1282X (SEQ ID NO: 8) is inserted in frame between lysine-81 and trptophan-82. FIG. 3B shows growth curves demonstrating that the 33 bp in-frame insertion shown in FIG. 3A had a minor effect on the functionality of the LEU2 gene. The indicated logarithmic samples were grown in a medium lacking leucine. The growth rate was assessed using a TECAN microplate reader, by measuring the optical density (O.D 600 nm) every 30 minutes for 50 hrs. 4743 LEU2 WT represents cells expressing the wild type LEU2 gene. leu2-CF,W1282X, and leu2-CF,W1282 represents strains expressing the LEU2 gene with the in-frame insertions described in FIG. 3A, with and without a stop codon, respectively.

### DESCRIPTION OF SPECIFIC EMBODIMENTS OF THE INVENTION

The present invention, in some embodiments thereof, relates to compositions and methods for identifying antisense guide RNA for RNA editing.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Adenosine-to-inosine RNA editing effected by the adenosine deaminase enzyme, ADAR, increases the repertoire of genome encoded RNAs. ADAR is a multi-domain protein, comprising a recognition domain and a catalytic domain. Steering ADAR to specific sites at selected transcripts, a strategy called site-directed RNA editing, holds great promise for the treatment of disease and as a tool to study protein and RNA function. One strategy developed for site-directed RNA editing is to create substrates around a target adenosine that are recognized by ADAR or an engineered ADAR. Essentially, these structures are generated by delivering antisense guide RNA oligos that create editable structures in trans. Currently, the most effective guide RNAs are composed of two essential elements: an antisense portion that is imperfectly complimentary to the mRNA in the vicinity of the targeted adenosine and a recruitment element to nucleate ADAR binding. Still, there are no generic rules for the construction of either element.

Whilst reducing the present invention to practice, the present inventors have now established a yeast-based screening system to determine ADAR activity. Consequently, specific embodiments disclose that this system can be used as a high throughput platform to identify guide RNA sequences suitable for site-directed RNA editing.

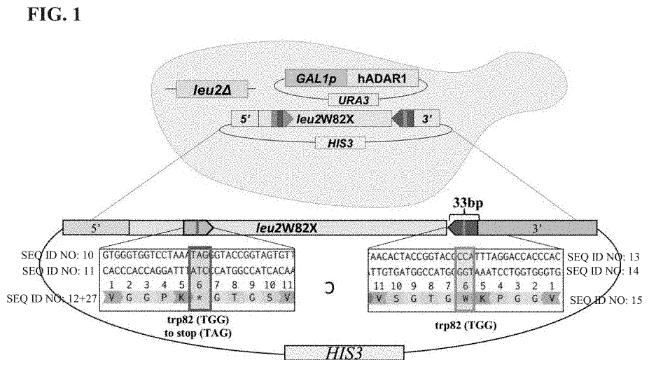

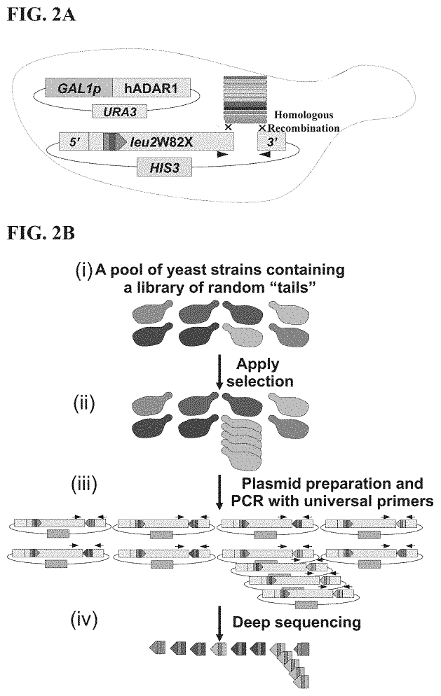

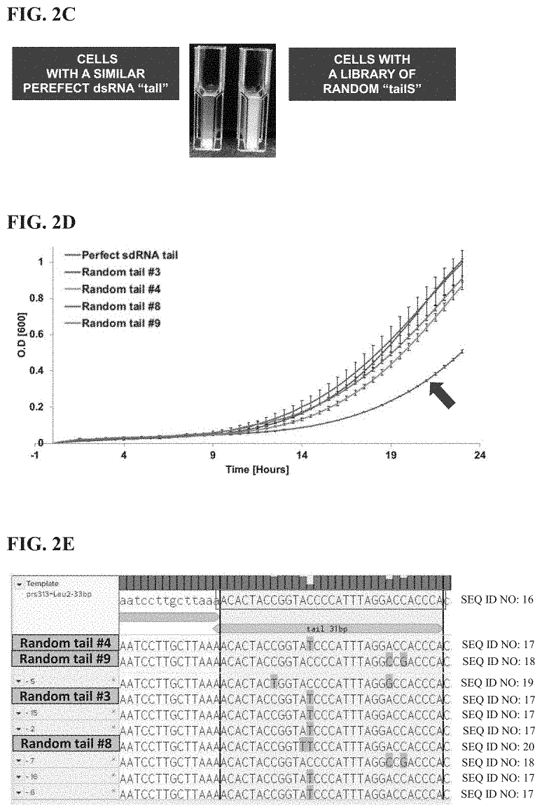

As is illustrated hereinunder and in the examples section, which follows, the present inventors developed a screening method based on a leucine auxotroph yeast strain which also harbors a plasmid that can conditionally express a human ADAR (Example 1, FIG. 1). In this strain, the endogenous LEU2 is deleted and replaced by a plasmid based dysfunctional LEU2 gene resulting from an in-frame nonsense mutation. In addition, the 3′ end of the dysfunctional LEU2 gene is followed by a replicable “tail” having complementarity to the regions flanking the nonsense mutation. Hence, when the “tail” folds back, a dsRNA structure is generated around the nonsense mutation. The activity of human ADAR can convert the adenosine in the in-frame inserted stop codon to inosine thereby enabling translation of a functional LEU2 and subsequently growth in a conditional medium lacking leucine (Example 1 FIG. 2D). Thus, in this screening method the rate of growth reflects the efficiency of editing. Utilizing this system, the suitability of “tails” of varied sequences to affect ADAR activity is evaluated (Example 1, FIGS. 2A-E).

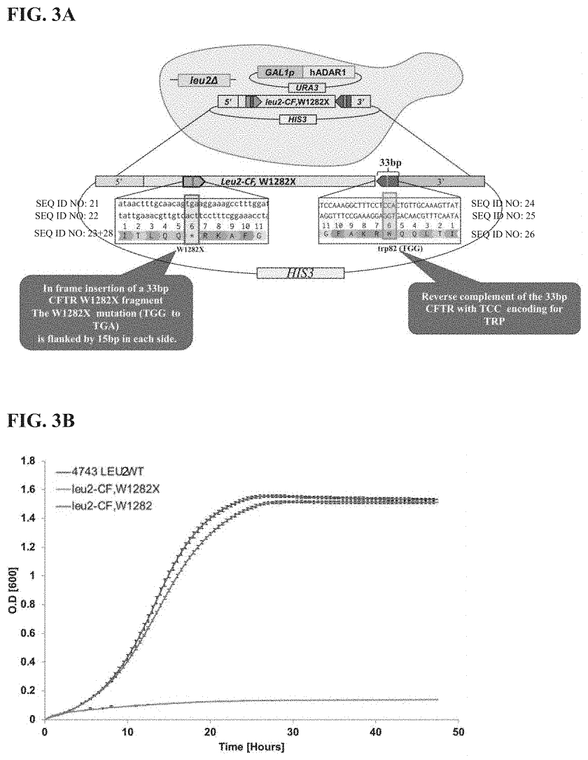

It is further contemplated that the screening method can be used to design better guides for directing ADAR to known mutations e.g. premature stop mutations which can be repaired by adenosine to inosine ADAR mediated RNA editing. To this end, the e.g. LEU2 gene comprises a heterologous fragment containing the mutation in a manner that introduces an in-frame premature stop codon, such that ADAR mediated editing of the mutation within the heterologous fragment enables the synthesis of a functional reporter protein. Thus, for example, as is illustrated hereinunder and in the examples section, which follows, a fragment that contains the CFTR W1282X nonsense mutation is inserted in frame between lysine81 and trptophan82 of the plasmid based LEU2, and “tails” of varied sequences are tested for their effect on ADAR activity by determining the rate of growth (Example 2, FIGS. 3A-B).

Thus, according to a first aspect of the present invention, there is provided a polynucleotide comprising:
(iii) a nucleic acid sequence encoding a reporter polypeptide comprising a heterologous nucleic acid sequence introducing an in-frame premature stop codon comprising an adenosine preventing translation of a functional reporter polypeptide; and
(iv) an additional nucleic acid sequence heterologous to said reporter polypeptide having at least 60% complementarity to said nucleic acid sequence comprising said in-frame premature stop codon;
   wherein said (i) and said (ii) are transcribed as a single transcript; and
   wherein conversion of said adenosine to inosine by RNA editing enables translation of a functional reporter polypeptide.

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

The term “nucleotide” refers to the respective nucleobase-(deoxy)ribosyl-phospholinker, as well as any chemical modifications of the ribose moiety or the phospho group. Thus, according to some embodiments, the nucleotide includes a locked ribosyl moiety (comprising a 2′-4′ bridge, comprising a methylene group or any other group, well known in the art), a nucleotide including a linker comprising a phosphodiester, phosphotriester, phosphoro(di)thioate, methylphosphonates, phosphoramidate linkers, or the like.

The polynucleotide of some embodiments of the invention comprises a nucleic acid sequence referred to as “(i)” and a nucleic acid sequence referred to as “(ii)” which are transcribed as a single transcript. That is the transcription of (i) and (ii) share the same transcription start site and end site.

The nucleic acid sequence (i) can be upstream or downstream to the nucleic acid sequence (ii).

According to specific embodiments, the nucleic acid sequence (i) is upstream of the nucleic acid sequence (ii).

The nucleic acid sequences (i) and (ii) can be separated using any nucleic acid linker between (i) and (ii) or they can be devoid of a nucleic acid linker.

According to specific embodiments, the polynucleotide is devoid of a nucleic acid linker between nucleic acid sequence (i) and nucleic acid sequence (ii).

As used herein, the term “reporter polypeptide” refers to a polypeptide which translation can be detected and optionally measured. Various types of reporter polypeptides and methods for the detection or measurement of their translation are well known to those of skill in the art. These include, but are not limited to, fluorescent proteins such as those derived from algae or synthetic versions thereof GFP (green fluorescent protein), YFP (yellow fluorescent protein), BFP (blue fluorescent protein), CFP (cyan fluorescent protein) and the like, lacZ, luxABCDE, luxAB, lucFF, uidA, RCFPs (Reef Coral Fluorescent Proteins), phoA, horseradish peroxidase (HPR), beta-galactosidase, alkaline phosphatase (AP) and a selectable polypeptide.

Translation of a functional reporter polypeptide can be monitored by a method appropriate to the particular reporter system used, including, but not limited to, visual imaging, fluorescence, radiography, flow cytometry, ELISA, enzyme-linked immunohistochemical assay, growth under selection conditions and others. For example, absorbance is measured for lacZ, luminescence is measured for luxABCDE, fluorescence is measured for GFP and growth under selection conditions is measured for selectable polypeptides.

The term “selectable polypeptide” is used herein to describe a polypeptide that can be used to select for a cell or cells containing the selectable polypeptide. Such selectable polypeptides are known in the art. Thus, for example, the selectable polypeptide may confer resistance to a selection agent such as e.g. an antibiotic or herbicide; may be able to neutralize or inactivate a toxic selection agent and protects the host cell from the agent's lethal or growth-inhibitory effects; other selectable polypeptides known as auxotrophic polypeptides complement a growth-inhibitory deficiency in the cell under certain conditions.

According to specific embodiments, the reporter polypeptide confers resistance to an antibiotic.

Such reporter polypeptides are well known in the art and include polypeptides conferring resistance to bleomycin family of antibiotics, puromycin, blasticidin, hygromycin, an aminoglycoside antibiotic [e.g. Kanamycin, Streptomycin, Gentamicin, Tobramycin, G418 (Geneticin), Neomycin B (Framycetin), Sisomicin, Amikacin, Isepamicin and the like], methotrexate, methionine sulphoximine.

According to specific embodiments, the polypeptide conferring resistance to an antibiotic is selected from the group consisting of KanMX, NatMX and HygB which confer resistance to the antibiotics geneticin (G418), nourseothricin (clonNAT) and hygromycin B (HygB), respectively.

According to specific embodiments, the reporter polypeptide is an auxotrophic polypeptide.

As used herein the term “auxotrophic polypeptide” refers to a reporter polypeptide required for synthesis of a nutritional metabolite essential for growth of a cell. That is, to enable growth in the absence of a functional auxotrophic polypeptide the cell requires exogenously adding the metabolite.

Such reporter polypeptides are well known in the art and include, but are not limited to, LEU2, TRP1, ADE2, LYS2 and cystathionine gamma-lyase.

According to specific embodiments, the auxotrophic polypeptide is selected from the group consisting of LEU2, TRP1, ADE2 and LYS2.

According to specific embodiments, the auxotrophic polypeptide is LEU2.

“LEU2 (3-isopropylmalate dehydrogenase)”, E.C. No. 1.1.1.85, refers to the polypeptide expression product of the LEU2 gene (Saccharomyces genome data base (SGD) systematic name: YCL018W, Gene ID 850342. LEU2 catalyzes the oxidation of 3-carboxy-2-hydroxy-4-methylpentanoate (3-isopropylmalate) to 3-carboxy-4-methyl-2-oxopentanoate. LEU2 is required for the biosynthesis of the amino acid leucine.

According to specific embodiments, the LEU2 is a yeast LEU2, such as provided in the following GenBank Accession No. NP_009911.

A non-limiting example of a nucleic acid sequence encoding LEU2 is provided in GenBank Accession No. NM_001178665 or SEQ ID NO: 4.

According to specific embodiments, the auxotrophic polypeptide is TRP1.

“TRP1 (Tyrosinase-related protein 1)”, E.C. No. 1.14.18, refers to the polypeptide expression product of the TRP1 gene (SGD systematic name: YDR007W, Gene ID 851570). TRP1 catalyzes the oxidation of 5,6-dihydroxyindole-2-carboxylic acid (DHICA) into indole-5,6-quinone-2-carboxylic acid in the presence of bound Cu(2+) ions. TRP1 is required for the biosynthesis of the amino acid tryptophan.

According to specific embodiments, the TRP1 is a yeast TRP1, such as provided in the following GenBank Accession No. NP_010290.

A non-limiting example of a nucleic acid sequence encoding TRP1 is provided in GenBank Accession No. NM_001180315 or SEQ ID NO: 5.

According to specific embodiments, the auxotrophic polypeptide is ADE2.

“ADE2 (phosphoribosylaminoimidazole carboxylase)”, E.C. No. 4.1.1.21, refers to the polypeptide expression product of the ADE2 gene (SGD systematic name: YDR007W Gene ID 854295). ADE2 catalyzes the conversion of 5′-phosphoribosyl-5-aminoimidazole (“AIR”) into 5′-phosphoribosyl-4-carboxy-5-aminoimidazole (“CAIR”). ADE2 is required for the biosynthesis of the amino acid adenine.

According to specific embodiments, the ADE2 is a yeast ADE2, such as provided in the following GenBank Accession No. NP_014771.

A non-limiting example of a nucleic acid sequence encoding ADE2 is provided in GenBank Accession No. NM_001183547 or SEQ ID NO: 6.

According to specific embodiments, the auxotrophic polypeptide is LYS2.

“LYS2 (L-2-aminoadipate reductase)”, E.C. No. 1.2.1.31, refers to the polypeptide expression product of the LYS2 gene (SGD systematic name: YDR007W, Gene ID 852412). LYS2 catalyzes the reduction of alpha-aminoadipate to alpha-aminoadipate 6-semialdehyde. LYS2 is required for the biosynthesis of the amino acid lysine.

According to specific embodiments, the LYS2 is a yeast LYS2, such as provided in the following GenBank Accession No. NP_009673.

A non-limiting example of a nucleic acid sequence encoding LYS2 is provided in GenBank Accession No. NM_001178463 or SEQ ID NO: 7.

As mentioned, the nucleic acid sequence (i) encodes a reporter polypeptide comprising a heterologous nucleic acid sequence introducing an in-frame premature stop codon comprising an adenosine.

According to specific embodiments, the in-frame premature stop codon is UAG or TAG.

According to a specific embodiment, the in-frame premature stop codon is UAG.

As used herein, the term “heterologous to the reporter polypeptide” refers to a sequence which is not native to the reporter polypeptide at least in localization or is completely absent from the native sequence of the reporter polypeptide.

According to specific embodiments, the heterologous nucleic acid sequence introducing an in-frame premature stop codon is a specific nucleic acid sequence of a gene associated with a disease.

As used herein, the phrase “a specific nucleic acid sequence of a gene associated with a disease” refers to a nucleic acid sequence alteration (i.e., mutation) which drives onset and/or progression of the disease, wherein this alteration can be repaired by RNA editing. According to specific embodiments, the mutation results in an in-frame stop codon in the gene associated with the disease. Non-limiting examples of such mutations include mutations in the CFTR gene (e.g. G542X; W1282X; R553X; 1162X; Y122X) associated with cystic fibrosis; W23X mutation in the low-density lipoprotein receptor (LDLR) associated with familial hypercholesterolaemia; mutations in Factor IX (e.g. E27K, G60S, R248Q) associated with Haemophilia-B; G269S mutation in the hexosaminidase A enzyme associated with Tay□ Sachs, and mutations in the ATM gene (e.g. G2250A, G3676A, R2032K) associated with ataxia telangiectasia.

According to a specific embodiment, the gene is CFTR.

According to a specific embodiment, specific nucleic acid sequence of a gene associated with a disease comprises the CFTR W1282X nonsense mutation.

According to other specific embodiments, the mutation in itself does not result in an in-frame stop codon in the gene associated with the disease; however a frameshift in the sequence comprising the mutation may introduce an in-frame stop codon. In this case the heterologous nucleic acid sequence is inserted to the nucleic acid encoding the reporter polypeptide by changing the frame of the gene associated with the disease such that an in-frame premature stop codon will prevent translation of a functional reporter polypeptide. Thus, for example, a disease associated with a mutation of Met (ATG) to ILE (ATA) that is followed by ASP (GAC) can be inserted as the heterologous sequence in another frame, thereby introducing XXA TAG ACX (instead of ATA GAC).

According to specific embodiments, the heterologous nucleic acid sequence introducing an in-frame premature stop codon is 15-120, 15-100, 20-100, 20-80, 20-50 nucleic acids long.

According to specific embodiments, the heterologous nucleic acid sequence introducing an in-frame premature stop codon is 15-120 nucleic acids long.

According to specific embodiments, the heterologous nucleic acid sequence introducing an in-frame premature stop codon is 25-40 nucleic acids long.

According to specific embodiments, the heterologous nucleic acid sequence introducing an in-frame premature stop codon is 30-36 nucleic acids long.

According to specific embodiments, the heterologous nucleic acid sequence introducing an in-frame premature stop codon is about 33 nucleic acids long.

According to specific embodiments, the heterologous nucleic acid sequence introducing an in-frame premature stop codon is 33 nucleic acids long.

The presence of the in-frame premature stop codon prevents translation of a functional reporter polypeptide such that conversion of the adenosine in the premature stop codon to inosine by RNA editing enables translation of a functional reporter polypeptide.

The skilled in the art knows how to design such a reporter polypeptide. Thus, for Example, a 33 bp nucleic acid sequence of CFTR comprising mutation W1282X (SEQ ID NO: 8) can be introduced between positions 244 and 246 corresponding to the LEU2 nucleic acid sequence as set forth in SEQ ID NO: 4, i.e. between lysine-81 and trptophan-82. Another non-limiting possibility is between positions 469 and 471 corresponding to the LEU2 nucleic acid sequence as set forth in SEQ ID NO: 4, i.e. between aspartic acid 158 alanine 156.

Thus, according to specific embodiments, when the reporter polypeptide is LEU2, the heterologous nucleic acid sequence introducing the in-frame premature stop codon is located between positions 244 and 246 corresponding to the LEU2 nucleic acid sequence as set forth in SEQ ID NO: 4.

Such adenosine to inosine conversion is typically performed by ADAR (adenosine deaminase acting on RNA).

Thus, according to specific embodiments, the polynucleotide comprises an additional nucleic acid sequence (iii) encoding ADAR.

Nucleic acid sequences (i)+(ii)+(iii) can be expressed as a single transcript or as two separate transcripts, one comprising (i)+(ii) and the other comprising (iii). Methods of expressing two distinct transcripts from a single polynucleotide are well known in the art and are further provided infra.

Alternatively, according to an aspect of the present invention there is provided a nucleic acid system comprising the polynucleotide comprising (i) and (ii) and a separate polynucleotide comprising a nucleic acid sequence encoding ADAR.

“ADAR (adenosine deaminase acting on RNA)”, E.C. No. 3.5.4, refers to the polypeptide expression product of the ADAR gene (Gene ID 103). ADAR catalyze the conversion of adenosine (A) to inosine (I) by hydrolytic deamination. Typically, ADARs share a common modulator organization which consists of a variable N-terminal region, a double stranded RNA binding domain and a zinc containing catalytic domain. Accordingly, the ADAR may be ADAR 1, 2 or 3.

According to specific embodiments, the ADAR is ADAR1.

According to specific embodiments the ADAR is ADAR2.

In some embodiments, the adenosine deaminase is derived from one or more metazoa species, including but not limited to, mammals, birds, frogs, squids, fish, flies and worms.

According to specific embodiments, the ADAR is a human ADAR (e.g., hADAR1 and hADAR2).

Non-limiting exemplary sequences of human ADAR are provided in the following GenBank Accession Numbers: NP_001020278, NP_001102, NP_001180424, NP_056655 and NP_056656

Non-limiting examples of nucleic sequence encoding human ADAR are provided in the following GenBank Accession Numbers: NM_001025107, NM_001111, NM_001193495, NM_015840 and NM_015841.

According to specific embodiments, the nucleic acid sequence encoding human ADAR comprises SEQ ID NO: 9.

According to specific embodiments, the nucleic acid sequence encoding human ADAR consists of SEQ ID NO: 9.

Any coding sequence of a reporter polypeptide or ADAR also encompasses functional isoforms and homologues (naturally occurring or synthetically/recombinantly produced), which exhibit the desired activity as described herein. Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide sequence provided herein; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same.

Sequence identity or homology can be determined using any protein or nucleic acid sequence alignment algorithm such as Blast, ClustalW, and MUSCLE.

The homolog may also refer to an ortholog, a deletion, insertion, or substitution variant, including a conservative and non-conservative amino acid substitution, as further described hereinbelow.

As mentioned, the polynucleotide of some embodiments of the invention comprises a nucleic acid sequence (ii) which is heterologous to the reporter polypeptide having at least 60% complementarity to the nucleic acid sequence comprising said in-frame premature stop codon.

According to specific embodiments, the nucleic acid sequence (ii) has at least 60% complementarity to the heterologous nucleic acid sequence introducing the in-frame premature stop codon comprised in nucleic acid sequence (i).

The nucleic acid sequence (ii) should have sufficient overlap and complementarity to the nucleic acid sequence (i) comprising the in-frame stop codon to allow for sequence specific hybridization of the nucleic acid sequence (ii) with the nucleic acid sequence (i) comprising the in-frame stop codon. The length and the % complementarity may be routinely determined by a person having ordinary skill in the art. In general, longer sequences provide more specificity—and consequently fewer off-target effects, e.g. through non-specific binding—and stronger binding to the target site.

According to specific embodiments, nucleic acid sequence (ii) is 15-120, 15-100, 20-100, 20-80, 20-50 nucleic acids long.

According to specific embodiments, nucleic acid sequence (ii) is 15-120 nucleic acids long.

According to specific embodiments, nucleic acid sequence (ii) is 25-40 nucleic acids long.

According to specific embodiments, nucleic acid sequence (ii) is 30-36 nucleic acids long.

According to specific embodiments, nucleic acid sequence (ii) is about 33 nucleic acids long.

According to specific embodiments, nucleic acid sequence (ii) is 33 nucleic acids long.

According to specific embodiments, nucleic acid sequence (ii) is about the same length as the heterologous nucleic acid sequence introducing an in-frame premature stop codon.

According to specific embodiments, nucleic acid sequence (ii) is the same length as the heterologous nucleic acid sequence introducing an in-frame premature stop codon.

As used herein, the term “complementarity” refers to base pair complementation e.g., A-T/U and C-G.

As used herein, “complementarity” refers to global complementarity, i.e., a complementarity over the entire nucleic acid sequence (i) having about the same length as nucleic acid sequence (ii) disclosed herein and not over portions thereof.

According to specific embodiments, the complementarity is over the heterologous nucleic acid sequence introducing the in-frame premature stop codon comprised in nucleic acid sequence (i).

According to specific embodiments, the nucleic acid sequence (ii) has at least 60%, at least 65%, at least 70%, at least 75%, at least 80%, at least 85%, at least 90%, at least 95% complementarity to the nucleic acid sequence comprising said in-frame premature stop codon.

According to a specific embodiment, the nucleic acid sequence (ii) has at least 60% complementarity to the nucleic acid sequence comprising said in-frame premature stop codon.

According to a specific embodiment, the nucleic acid sequence (ii) has at least 70% complementarity to the nucleic acid sequence comprising said in-frame premature stop codon.

According to a specific embodiment, the nucleic acid sequence (ii) has at least 80% complementarity to the nucleic acid sequence comprising said in-frame premature stop codon.

The specificity of ADAR can be increased to only convert adenosine comprised in the in-frame stop codon by providing a nucleic acid sequence (ii) that comprises a mismatch opposite the adenosine in the premature stop codon in nucleic acid sequence (i). The mismatch can be created by providing a nucleic acid sequence (ii) having a cytidine or uridine, according to a specific embodiment a cytidine, opposite the adenosine in the premature stop codon in nucleic acid sequence (i). Upon deamination of the adenosine in the premature stop codon in nucleic acid sequence (i), the nucleic acid sequence (i) will obtain an inosine which, for most biochemical processes, is “read” by the cell's biochemical machinery as a guanosine. Hence, following adenosine to inosine conversion, the mismatch is resolved (as inosine is capable of base pairing with the opposite cytidine in the nucleic acid sequence (ii)).

Thus, according to specific embodiments, the nucleic acid sequence (ii) comprises a mismatch with the adenosine in the premature stop codon in nucleic acid sequence (i).

According to specific embodiments, the nucleic acid sequence (ii) comprises a cytidine opposite the adenosine to be edited.

Any non-specific editing of adenosines can be limited, by making sure that the adenosines that should not be edited, or at least at a lower frequency, encounter an opposite nucleotide with a 2′-0 modified ribose moiety, such as a 2′-OMe, as the latter is known to reduce the efficiency of editing of the opposite adenosine. Hence, according to specific embodiments, in cases where over-editing is to be avoided, the nucleic acid sequence (ii) may be chemically modified.

According to specific embodiments, the nucleic acid sequence (ii) comprises 2′-O methyl groups in positions which oppose adenosines when the nucleic acid sequence (ii) is paired to the nucleic acid sequence (i) if these adenosines in the nucleic acid sequence (i) is not a target for editing.

It is envisaged that other 2′-0 substitutions of the ribosyl moiety, such as 2′-methoxyethyl (2′-MOE) and 2′-O-dimethylallyl groups may also reduce unwanted editing of the corresponding (opposite) the adenosine in the in-frame stop codon. Other chemical modifications are readily available to the person having ordinary skill in the art of oligonucleotide synthesis and design. The synthesis of such chemically modified oligonucleotide constructs and testing them in methods according to the invention does not pose an undue burden and other modifications are encompassed by the present invention.

Alternatively, or additionally, an opposing base being a guanine or adenine may be provided, as these nucleobases generally impede deamination of the opposing base.

According to other specific embodiments, nucleic acid sequence (ii) is not chemically modified.

To express the polynucleotides and/or the polynucleotide systems disclosed herein using recombinant technology, the polynucleotides may be ligated into a nucleic acid expression construct, under the transcriptional control of a cis-regulatory sequence (e.g., promoter sequence) suitable for directing constitutive or inducible transcription of the polynucleotide sequence in a cell.

Thus, according to an aspect of the present invention, there is provided the polynucleotide or the system, wherein the polynucleotide is comprised in a nucleic acid construct comprising a cis-acting regulatory element for directing expression of the polynucleotide.

According to specific embodiments, the regulatory element is a heterologous regulatory element.

The nucleic acid construct (also referred to herein as an “expression vector”) of some embodiments of the invention includes additional sequences which render this vector suitable for replication and integration in prokaryotes, eukaryotes, or preferably both (e.g., shuttle vectors).

In addition, a typical cloning vector may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

According to specific embodiments, the promoter utilized by the nucleic acid construct of some embodiments of the invention is active in the specific cell population transformed.

According to specific embodiments, the promoter utilized by the nucleic acid construct of some embodiments of the invention is an inducible promoter such as, but not limited to galactose inducible promoter (GAL1-1 promoter) or the copper induced promoter (CUP1-1 promoter).

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

It will be appreciated that the individual elements comprised in the expression vector can be arranged in a variety of configurations. For example, enhancer elements, promoters and the like, and even the polynucleotide sequence(s) can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

Non-limiting examples of bacterial constructs include the pET series of E. coli expression vectors [Studier et al. (1990) Methods in Enzymol. 185:60-89).

In yeast, a number of vectors containing constitutive or inducible promoters can be used, as disclosed in U.S. Pat. No. 5,932,447. Alternatively, vectors can be used which promote integration of foreign DNA sequences into the yeast chromosome.

In cases where plant expression vectors are used, the expression of the coding sequence can be driven by a number of promoters. For example, viral promoters such as the 35S RNA and 19S RNA promoters of CaMV [Brisson et al. (1984) Nature 310:511-514], or the coat protein promoter to TMV [Takamatsu et al. (1987) EMBO J. 6:307-311] can be used. Alternatively, plant promoters such as the small subunit of RUBISCO [Coruzzi et al. (1984) EMBO J. 3:1671-1680 and Brogli et al., (1984) Science 224:838-843] or heat shock promoters, e.g., soybean hsp17.5-E or hsp17.3-B [Gurley et al. (1986) Mol. Cell. Biol. 6:559-565] can be used. These constructs can be introduced into plant cells using Ti plasmid, Ri plasmid, plant viral vectors, direct DNA transformation, microinjection, electroporation and other techniques well known to the skilled artisan. See, for example, Weissbach & Weissbach, 1988, Methods for Plant Molecular Biology, Academic Press, NY, Section VIII, pp 421-463.

Other expression systems such as insects and mammalian host cell systems which are well known in the art can also be used by some embodiments of the invention.

The type of vector used by some embodiments of the invention will depend on the cell type transformed. The ability to select suitable vectors according to the cell type transformed is well within the capabilities of the ordinary skilled artisan and as such no general description of selection consideration is provided herein.

Thus, as non-limiting examples, for expression in yeast, the yeast centromeric plasmid system (Genetics. 1989 May; 122(1):19-27 PMID: 2659436), or the “Gateway recombination cloning technology” (Invitrogen), can be used.

Various methods can be used to introduce the expression vector of some embodiments of the invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

The cell may be transformed stably or transiently with the nucleic acid constructs disclosed herein. In stable transformation, the nucleic acid molecule is integrated into the cell genome and as such it represents a stable and inherited trait. In transient transformation, the nucleic acid molecule is expressed by the cell transformed but it is not integrated into the genome and as such it represents a transient trait.

The present invention also contemplates cells comprising the polynucleotides, systems and constructs.

Thus, according to an aspect of the present invention there is provided a cell expressing the polynucleotide or the system disclosed herein.

According to specific embodiments, the cell may be a prokaryotic or a eukaryotic cell.

According to specific embodiments, the cell is a eukaryotic cell.

Non-limiting examples of eukaryotic cells which may be used with some embodiments of the invention include but are not limited to, mammalian cells, fungal cells, yeast cells, insect cells, algal cells or plant cells.

According to specific embodiments, the cell is a yeast cell.

Non-limiting examples of yeasts that can be used with specific embodiments of the invention include Saccharomyces cerevisiae and Schizosaccharomyces pombe.

According to specific embodiments, the yeast is Saccharomyces cerevisiae.

According to specific embodiments, the cell is not a bacterium.

According to specific embodiments, the cell is not E. coli.

According to specific embodiments, the cell is a cell in which an endogenous or an exogenous ADAR is capable of editing RNA in.

Thus, according to specific embodiments, the cell expresses an endogenous ADAR.

According to specific embodiments, the cell does not express an endogenous ADAR.

According to specific embodiments, the cell expresses an exogenous ADAR.

As shown in the Examples section which follows, the present inventors established a yeast-based screening system to determine ADAR activity which can be used e.g. as a high-throughput platform to identify guide RNA sequences suitable for site-directed RNA editing.

Thus, according to an aspect of the present invention there is provided a method of identifying an antisense suitable for site-directed RNA editing, the method comprising determining in the cell disclosed herein translation of said functional reporter polypeptide, wherein when said cell is not expressing an ADAR capable of editing RNA in said cell the method comprises expressing in said cell a polynucleotide comprising a nucleic acid sequence encoding ADAR capable of editing RNA in said cell prior to said determining, wherein said translation above a predetermined threshold indicates said (ii) is a suitable antisense for site-directed RNA editing of said in-frame premature stop codon.

The method may be effected in-vivo, in-vitro or ex-vivo.

According to specific embodiments, the method is effected in-vitro or ex-vivo.

According to specific embodiments, the method is effected in-vivo.

As used herein the phrase “predetermined threshold” refers to at least a minimal detectable level e.g., by optical density or fluorescence assay, of translation of a functional reporter polypeptide.

According to specific embodiment, the predetermined threshold is a significant detectable level of translation of a functional reporter polypeptide.

According to specific embodiments, the predetermined threshold is the level of translation of a functional reporter polypeptide wherein nucleic acid sequence (ii) has perfect complementarity to the nucleic acid sequence comprising said in-frame premature stop codon with the exception of a mismatch opposite the adenosine in the premature stop codon.

Translation of a functional reporter polypeptide may be determined by e.g. growth under selection conditions, visual inspection, fluorescence, radiography, flow cytometry, ELISA, enzyme-linked immunohistochemical assay, depending on the reporter polypeptide used. Thus, according to specific embodiments, the reporter polypeptide is an auxotrophic polypeptide or a polypeptide conferring resistance to an antibiotic, and the determining is effected by determining growth and/or survival under selective conditions.

Thus, according to specific embodiments, when the reporter polypeptide is an auxotrophic polypeptide or a polypeptide conferring resistance to an antibiotic, the predetermined level is reflected by an optical density of at least 0.4 following growth for at least 15 hours under selective conditions.

The method disclosed herein may be used in high throughput screening systems (in arrayed format) for testing a large variety of e.g. antisense sequences.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

As used herein the term “about” refers to ±10%

The terms “comprises”, “comprising”, “includes”, “including”, “having” and their conjugates mean “including but not limited to”.

The term “draw” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Materials and Methods

Yeast strains—All the strains used in this study are isogenic to the diploid strain BY4743 (MATa/α ura3 Δ0/ura3 Δ0 leu2 Δ0/leu2 Δ0 his3 Δ1/his3 Δ1 lys2 Δ0/LYS2 met15 Δ0/MET15) (17).

Growth conditions—Yeast cells were grown at 30° C. in synthetic complete (0.17% Yeast nitrogen base w/o aa and Ammonium Sulfate, 0.1% Glutamic acid, supplemented with either 2% glucose (SD), or galactose (SC-GAL), and 0.2% of either: (-Uracil-Histidine) or (Uracil-Histidine-Leucine) amino acid mix.

Plasmids—The “Gateway recombination cloning technology” (Invitrogen), was used to clone the human ADARs (ADAR1, or ADAR2) into the URA3 marked plasmid, pYES2-DEST52 Gateway destination vector (Cat #12286-019). This plasmid enables the conditional expression of the human ADARs in yeast, under a galactose inducible promoter (GAL1p-hADAR1). The plasmid carrying the LEU2 reporter gene was created by ligating a XhoI/XbaI PCR fragment of the yeast LEU2 gene (including 408 bp of its 5′ promoter region, and 358 bp of the 3′ UTR) into the HIS3 marked plasmid pRS313 (Sikorski R S and Hieter P. Genetics. 1989; 122(1):19-27), digested with XhoI and XbaI. Site directed mutagenesis was used to introduce the BamHI restriction site immediately after the LEU2 stop codon. This restriction site was used to linearize the plasmid and enabled the insertion of the “random tails” library by homologous recombination.

Oligos—The single stranded 70 bps oligos synthesized by IDT comprised the 33pb random “tails” (denoted as 33×); flanked by 20 bps universal sequences at their 3′ and 5′ ends, which serve as template for PCR amplification (i.e. 5′-TTAAGAAAATCCTTGCTTAA-33x-AAAGATTCTCTTTTTTTATG-3′, SEQ ID NO: 1).

The following forward (f) and reverse (r) primers were used to PCR amplify the single stranded oligos:

f:
(SEQ ID NO: 2)
5′-CCGAAGTCGGTGATGCTGTCGCCGAAGAAGttaagaaaatccttgct
taa-3′
r-
(SEQ ID NO: 3)
5′-ATTTCATTTATAAAGTTTATGTACAAATATcataaaaaaagagaatc
ttt-3′

The 30 bps region of the plasmid (denoted in uppercase letters) extending from the regions that anneal with the universal sequence (denoted in bold, lowercase letters) increase the homology to the regions flanking the BamHI restriction site on the plasmid based leu2 reporter gene, enabling the insertion of the PCR products by homologous recombination.

Yeast transformation—Yeast transformation with PCR products containing the random tails, and the BamHI linearized plasmid carrying the reporter gene were performed by electroporation, as previously described (Benatuil L, et al. Protein Eng Des Sel. 2010; 23(4):155-9.

Growth evaluation—Growth rate was assessed in a 96 wells plate using a TECAN instrument Spark 10M microplate reader, by measuring the optical density at a wavelength of 600 nm (O.D₍₆₆₀ₙₘ₎) every 30 minutes for 25 hours.

### Example 1

Development of a Yeast-Based Screening System for Finding an Optimal Guide-RNA for Site-Directed RNA Editing

The baker yeast Saccharomyces cerevisiae is an organism whose origins precede the emergence of ADARs, and thus does not express an endogenous ADAR or undergo editing. The present inventors have developed a high-throughput platform utilizing Saccharomyces cerevisiae that can be used to screen vast libraries of guide-RNA sequences in order to determine which sequences trigger editing and which do not. The screening method is based on a leucine auxotroph yeast strain, i.e., unable to grow in a medium without leucine, which harbors a plasmid that can conditionally express one of the human ADARs (hADAR) under a galactose inducible promoter (e.g. GAL1p-ADAR1 in FIG. 1). The endogenous LEU2 is deleted and replaced by a plasmid based LEU2, which is dysfunctional (e.g. leu2W82X), as a result of a nonsense mutation (the conversion of trp82 (W)-(TGG), to a stop (X) codon (TAG)-leu2W82X). In addition, the 3′ end of the leu2W82X gene is followed by a replicable “tail” that can fold back at the RNA level to create dsRNA (denoted by a blue arrow in FIG. 1). This “tail” represents the reverse complement (RC) sequence of trp82 (CCA), centered around 15 bp of the flanking region. When the “tail” folds back, a perfect dsRNA is generated around the leu2W82X mutation, with an exception of a single mismatch between A and C at the STOP codon-containing sequence (the result of UAG and CCA self-folding). The activity of hADAR can change the UAG to the UIG codon encoding tryptophan. If the RNA editing is efficient enough, the wild-type Leu2 protein generated will permit growth in a synthetic dropout liquid medium lacking leucine (SD-LEU) (FIG. 2D). The rate of growth reflects the efficiency of editing.

In order to establish a high-throughput screening system for identifying optimal guide-RNA for triggering ADRA RNA editing at the premature termination codon, a library of single-stranded oligos comprising semi random “tails” each having over 80% sequence identity with the flanking region of the premature STOP codon was synthesized (FIGS. 2A-E). These oligos were PCR amplified, and the products were co-transformed with the BamHI linearized plasmid carrying the leu2W82X gene, marked with the histidine3 (HIS3) gene (FIG. 2A). Plating on a synthetic dropout (SD) medium lacking uracil (hADAR1 plasmid selection) and histidine (encircled HIS3 plasmid) (SD-URA-HIS), enabled the selection of a library composed of 10⁸⁻10⁹ colonies, each containing a plasmid that is encircled, by a different “tail” via homologous recombination at the 3′ end of the engineered leu2W82X gene.

Following, leucine starvation growth conditions enabled enrichment of cells carrying tails that allow more efficient editing. To further enrich for such tails, the library was pooled, diluted to an OD600 of 0.1, and subjected to selection in a SC-GAL-URA-HIS-LEU medium. This medium is supplemented with 2% galactose (to enable GAL1p hADAR1 expression), and lacking uracil (hADAR1 plasmid selection), histidine (encircled HIS3 plasmid selection), and leucine (selection for hADAR1 mediated Leu2 protein synthesis). Three iterative rounds of enrichment were performed, which led to increased cell density in the sample containing the library, compared to the reference sample with cells carrying tails that form a perfect dsRNA with the target (FIG. 2C).

To isolate the cells that were enriched during the selection, a sample was collected from the SD-LEU media and plated on a non-selective rich media. The growth curves of selected colonies formed by the single cells showed an approximately 1.5-2-fold increase in their growth rate compared to the intermediate growth-rate baseline (FIG. 2D).

In order to identify the sequences of the “tails” that facilitated better growth rate, the plasmids were sequenced using primers flanking the tail insertion site (FIG. 2E). Retransformation of these plasmids into an independent strain expressing hADAR1, confirmed that the improvements in growth rate were the result of specific base substitutions within the tails, and not due to genomic mutations.

### Example 2

Utilizing the Yeast-Based Screening System to Select a Guide-RNA for a Known Mutation

The experimental system described in Example 1 hereinabove can be used according to specific embodiments to design better guides for directing ADAR to known premature stop mutations which can be repaired by A to-I ADAR mediated RNA editing. Non-limiting examples of such mutations include the CFTR nonsense mutants, e.g. G542X; W1282X; R553X; 1162X; Y122X causing cystic fibrosis; W23X mutation in the low-density lipoprotein receptor (LDLR) causing familial hypercholesterolaemia; E27K, G60S, R248Q mutations in Factor IX, causing Haemophilia-B; G269S mutation in the hexosaminidase A enzyme causing Tay□ Sachs, and the G2250A, G3676A, R2032K mutants in the ATM gene causing ataxia telangiectasia.

To this end, the reporter gene comprises a heterologous fragment containing the premature stop mutations described above, such that ADAR mediated editing of the mutation within the heterologous fragment enables the synthesis of a functional reporter protein.

Thus, for example, a 33 bp fragment that contains the CFTR W1282X nonsense mutation (see FIG. 3A), is inserted in frame between lysine81 and trptophan-82 of the plasmid based LEU2 (termed: leu2-CF, W1282X).

To allow testing the effect of the insertion on the functionality of the reporter gene and the expected maximum growth rate, a control plasmid is also created in which the stop codon within the 33 bp fragment is swapped with tryptophan. For example, such a replacement within the CF, W1282X insertion (CF,W1282) had a minor effect on the growth rate of the cells carrying this plasmid in SD-LEU (FIG. 3B).

In the next step, to identify guide-RNA sequences that improve the intermediate growth-rate baseline for each of the selected mutations, a random library of oligos is created and tested as described in Example 1 hereinabove.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

(Other References are Cited Throughout the Application)
1. Savva Y A, Rieder L E, Reenan R A. The ADAR protein family. Genome Biol. 2012; 13(12):252.
2. Nishikura K. Functions and regulation of RNA editing by ADAR deaminases. Annu Rev Biochem. 2010; 79:321-49.
3. Eisenberg E, Levanon E Y. A-to-I RNA editing—immune protector and transcriptome diversifier. Nat Rev Genet. 2018; 19(8):473-90.
4. Basilio C, Wahba A J, Lengyel P, Speyer J F, Ochoa S. Synthetic polynucleotides and the amino acid code. V. Proc Natl Acad Sci USA. 1962; 48:613-6.
5. Zinshteyn B, Nishikura K. Adenosine-to-inosine RNA editing. Wiley Interdiscip Rev Syst Biol Med. 2009; 1(2):202-9.
6. Mali P, Yang L, Esvelt K M, Aach J, Guell M, DiCarlo J E, et al. RNA-guided human genome engineering via Cas9. Science. 2013; 339(6121):823-6.
7. Komor A C, Badran A H, Liu D R. CRISPR-Based Technologies for the Manipulation of Eukaryotic Genomes. Cell. 2017; 168(1-2):20-36.
8. Hsu P D, Lander E S, Zhang F. Development and applications of CRISPR-Cas9 for genome engineering. Cell. 2014; 157(6):1262-78.
9. Montiel-Gonzalez M F, Diaz Quiroz J F, Rosenthal J J C. Current strategies for Site-Directed RNA Editing using ADARs. Methods. 2019; 156:16-24.
10. Wettengel J, Reautschnig P, Geisler S, Kahle P J, Stafforst T. Harnessing human ADAR2 for RNA repair—Recoding a PINK1 mutation rescues mitophagy. Nucleic Acids Res. 2017; 45(5):2797-808.
11. Montiel-Gonzalez M F, Vallecillo-Viejo I, Yudowski G A, Rosenthal J J. Correction of mutations within the cystic fibrosis transmembrane conductance regulator by site-directed RNA editing. Proc Natl Acad Sci USA. 2013; 110(45):18285-90.
12. Cox D B T, Gootenberg J S, Abudayyeh 00, Franklin B, Kellner M J, Joung J, et al. RNA editing with CRISPR-Cas13. Science. 2017; 358(6366):1019-27.
13. Merkle T, Merz S, Reautschnig P, Blaha A, Li Q, Vogel P, et al. Precise RNA editing by recruiting endogenous ADARs with antisense oligonucleotides. Nat Biotechnol. 2019; 37(2):133-8.
14. Katrekar D, Chen G, Meluzzi D, Ganesh A, Worlikar A, Shih Y R, et al. In vivo RNA editing of point mutations via RNA-guided adenosine deaminases. Nat Methods. 2019; 16(3):239-42.
15. Pokharel S, Beal P A. High-throughput screening for functional adenosine to inosine RNA editing systems. ACS Chem Biol. 2006; 1(12):761-5.
16. Garncarz W, Tariq A, Handl C, Pusch O, Jantsch M F. A high-throughput screen to identify enhancers of ADAR-mediated RNA-editing. RNA Biol. 2013; 10(2):192-204.
17. Brachmann C B, Davies A, Cost G J, Caputo E, Li J, Hieter P, et al. Designer deletion strains derived from Saccharomyces cerevisiae S288C: a useful set of strains and plasmids for PCR-mediated gene disruption and other applications. Yeast. 1998; 14(2):115-32.
18. Benatuil L, Perez J M, Belk J, Hsieh C M. An improved yeast transformation method for the generation of very large human antibody libraries. Protein Eng Des Sel. 2010; 23(4):155-9.

1. A polynucleotide comprising:
(i) a nucleic acid sequence encoding a reporter polypeptide comprising a heterologous nucleic acid sequence introducing an in-frame premature stop codon comprising an adenosine preventing translation of a functional reporter polypeptide; and
(ii) an additional nucleic acid sequence heterologous to said reporter polypeptide having at least 60% complementarity to said nucleic acid sequence comprising said in-frame premature stop codon;
wherein said (i) and said (ii) are transcribed as a single transcript; and
wherein conversion of said adenosine to inosine by RNA editing enables translation of a functional reporter polypeptide.

2. The polynucleotide of claim 1, wherein said reporter polypeptide is an auxotrophic polypeptide.

3. The polynucleotide of claim 2, wherein said auxotrophic polypeptide is selected from the group consisting of LEU2, TRP1, ADE2 and LYS2.

4. The polynucleotide of claim 1, wherein said reporter polypeptide confers resistance to an antibiotic.

5. (canceled)

6. The polynucleotide of claim 1, wherein said reporter polypeptide is LEU2.

7. The polynucleotide of claim 6, wherein said heterologous nucleic acid sequence introducing said in-frame premature stop codon is located between positions 244 and 246 corresponding to the LEU2 nucleic acid sequence as set forth in SEQ ID NO: 4.

8. The polynucleotide of claim 1, wherein said heterologous nucleic acid sequence introducing said in-frame premature stop codon is a specific nucleic acid sequence of a gene associated with a disease.

9. The polynucleotide of claim 8, wherein said gene is selected from the group consisting of CFTR, LDLR, Factor IX, hexosaminidase and ATM.

10. The polynucleotide of claim 1, wherein said heterologous nucleic acid sequence introducing said in-frame premature stop codon is 15-120 nucleic acids long.

11. (canceled)

12. The polynucleotide of claim 1, wherein said at least 60% complementarity is at least 80% complementarity.

13. The polynucleotide of claim 1, wherein said (ii) comprises a mismatch with said adenosine.

14. The polynucleotide of claim 1, wherein said (ii) is 15-120 nucleic acids long.

15. The polynucleotide of claim 1, wherein said (i) is upstream of said (ii).

16. The polynucleotide of claim 1, being devoid of a nucleic acid linker between said (i) and said (ii).

17. The polynucleotide of claim 1, comprising (iii) an additional nucleic acid sequence encoding ADAR.

18. A nucleic acid system comprising the polynucleotide of claim 1, and a polynucleotide comprising a nucleic acid sequence encoding ADAR.

19. (canceled)

20. A cell expressing the polynucleotide of claim 1.

21. The cell expressing the system of claim 18.

22-25. (canceled)

26. The cell of claim 21, wherein said cell is a yeast cell.

27. (canceled)

28. A method of identifying an antisense suitable for site-directed RNA editing, the method comprising determining in the cell of claim 20 translation of said functional reporter polypeptide, wherein when said cell is not expressing an ADAR capable of editing RNA in said cell the method comprises expressing in said cell a polynucleotide comprising a nucleic acid sequence encoding ADAR capable of editing RNA in said cell prior to said determining,
wherein said translation above a predetermined threshold indicates said (ii) is a suitable antisense for site-directed RNA editing of said in-frame premature stop codon.

29-34. (canceled)
